(19) United States
(12) Patent Application Publication
(10) Pub. No.: US 2017/0267965 A1
(43) Pub. Date: Sep. 21, 2017

(54) METHOD AND DEVICE FOR DETECTING INOCULATION AND AUTOMATED INOCULATION FACILITY PROVIDED WITH SUCH A DETECTION DEVICE

(71) Applicant: INTELLIGENCE ARTIFICIELLE APPLICATIONS, MONTPELLIER (FR)

(72) Inventors: Christian CUREL, LAVERUNE (FR); Michel ROCH, SAINT BRES (FR)

(21) Appl. No.: 15/528,691

(22) PCT Filed: Nov. 17, 2015
(86) PCT No.: PCT/FR2015/053095

§ 371 Date: May 22, 2017

(30) Foreign Application Priority Data
Nov. 26, 2014 (FR) 1461492

(51) Int. Cl. C12M 1/34; C12Q 3/00; C12M 1/12; C12M 1/36; C12M 1/26

(52) U.S. Cl. CPC C12M 41/36; C12M 41/48; C12M 33/00; C12M 25/06; C12Q 3/00; G06T 7/0012

(57) ABSTRACT

A method for detecting inoculation in order to detect inoculation with a sample of a solid culture medium (21) present in a layer on a culture plate (20), said culture plate (20) being positioned on a support (2), said method comprising a step of projecting an incident light flux towards said support (2) in order to allow oblique illumination of the surface referred to as the inoculation surface (22) of said culture medium (21) of the culture plate (20) when the culture plate (20) is provided in the disposed state on said support (2), a step of displaying, on a display area (41) of a display screen (4), a two-dimensional image of the incident light flux reflected by the inoculation surface (22) of the culture plate (20) in the illuminated state illuminated by the means (3) for projecting a light flux, and a step of photographing the display area (41) of the screen (4).

The present invention relates to a method and a device for detecting inoculation, as well as an automated inoculation facility outfitted with such a detection device.

In the fields of medical diagnostics and industrial, agricultural and food, pharmaceutical or cosmetic microbiological control, agar culture media on culture plates, particularly Petri dishes, have for decades constituted an essential tool for the detecting and identifying of microorganisms, possibly pathogenic ones.

The inoculation of such culture media is done in the classical manner, manually, with the aid of an inoculation tool which might be an eyelet, a loop generally made of platinum which needs to be heated for purposes of sterilization between two uses, a swab or a Trigalski ball. The inoculation can likewise be done with the aid of a disposable pipette. The inoculation can likewise be done in automatic fashion, using systems developed and marketed for this purpose.

The inoculation generally involves an operation or step of depositing a biological sample being analyzed on the surface of the solid culture medium, such as agar, and an operation or step of spreading this biological sample over said surface, with the aid of an inoculation tool. This spreading operation ensures a dilution of the sample.

This spreading operation may be done over said surface with the aid of an inoculation tool of the aforementioned type, which can move over said surface along a path depending on the chosen method of isolation. Thus, one of the best known techniques of isolation is the method of dial isolation, involving the forming of stripes by a predetermined methodology on the surface of said solid culture medium. Other methods such as the method of spiral isolation or the method of numbered isolation can also be used.

Depending on the inoculation tool used, the step of depositing the biological sample may be done at the same time or distinct from the step of spreading of the sample with the aid of said inoculation tool.

In the case of inoculation done with the aid of an automated inoculation device, there is a major risk of false negatives resulting from an absence of inoculation. This absence of inoculation may result either from the absence of a depositing of the sample on the inoculation surface of the culture medium, especially when the depositing and the isolation are done in two consecutive, distinct operations, or the absence of an isolation of the sample possibly resulting from a malfunctioning of the inoculation tool or a fault, such as the height of the solid culture medium.

The absence of deposition of the sample or the isolation necessarily results in an absence of growth of the microorganisms after incubation of the culture plate, so that a negative result is inferred. The consequences may be dramatic in the case of a false negative, that is, the absence of growth of microorganisms resulting from an absence of inoculation and not an absence of microorganisms in the sample.

One purpose of the present invention is thus to propose a method and a device for detecting inoculation as well as an inoculation facility whose design is able to prevent the presence of false negatives.

Another purpose of the present invention is to propose a method and a device for detecting inoculation as well as an inoculation facility whose design makes it possible to obtain precise images able to be processed, especially by basic image processing methods in a brief time.

Toward this end, the subject matter of the invention is a method for detecting inoculation in order to detect inoculation with a sample of a solid culture medium present in a layer on a culture plate, said culture plate being positioned on a support, characterized in that said method includes a step of projecting an incident light flux towards said support in order to allow oblique illumination of the surface referred to as the inoculation surface of said culture medium of the culture plate when the culture plate is provided in the disposed state on said support, a step of displaying on a display area of a display screen of a two-dimensional image of the incident light flux reflected by the inoculation surface of the culture plate in the illuminated state illuminated by the means for projecting a light flux comprising at least one illumination element and a step of photographing the display area of the screen, said photograph being able to be processed to determine the inoculation state of the culture medium.

The inoculation surface of the solid culture medium, and particularly in the case of an agar culture medium, is a smooth surface prior to inoculation. The inoculation, whether done by simultaneous or consecutive depositing and isolation, generates a modification in the state of said surface. Since the surface of the culture medium is smooth and relatively planar, the reflection of that surface may be approximated by a specular reflection. Therefore, it is easy to position the screen on the path taken by the beam of rays reflected by said inoculation surface and to obtain an extremely precise 2D image, where the features of excess thickness or hollows of the surface will appear dark or bright as compared to the planar zones which are more uniform. This modification results from the modification of the planarity of the agar surface which produces a modification of the angles of reflection of the incident light flux. As a result, it is possible to identify, especially in the case of an inoculation in two steps, both the deposition of the sample which appears as an excess thickness of the surface and a dark zone in that photograph, and the isolation of said sample which appears as a hollow feature of the surface and a bright feature in that photograph.

Preferably, said method involves a step of photographic processing as a function of reference data in order to determine the state of the inoculation (OK/not OK).

Preferably, said method also involves a step of storing said photographs and identification of said photographs in connection with a marking of the inoculated sample and/or the culture plate.

The invention also involves a device for detecting inoculation in order to detect inoculation with a sample of a solid culture medium present in a layer on a culture plate, characterized in that the device comprises a support to receive the culture plate being inoculated, means of projecting an incident light flux towards said support in order to allow oblique illumination of the surface referred to as the inoculation surface of said culture medium of the culture plate when the culture plate is in the disposed state on said support, a display screen comprising a display area able to display a two-dimensional image of the incident light flux reflected by the inoculation surface of the culture plate in the state illuminated by the means of projecting a light flux, and means of photographing the display area of the screen.

Thanks to the design of the device, besides the possibility of obtaining a precise 2D image for the reasons mentioned above, the device can be arranged above and at a distance from the support to allow an unimpeded displacement of the support.

Preferably, the device comprises a memory for storing said photographs of the display area of the screen and means of processing said photographs in order to determine the state of inoculation.

Preferably, the means of processing of said photographs comprise means of analyzing the gray levels of at least a portion of the photograph.

The portion of the photograph being analyzed in gray levels, known as the region of interest, corresponds to the portion of the photograph enabling a visualization of the zone of the inoculation surface whose surface appearance is liable to be modified by said inoculation. This modification of the surface may result from the presence of a drop of sample after the depositing of said sample on the inoculation surface, striations, or other items.

The image processing thanks to the simplicity and precision of the images can be done with the aid of known image processing software.

Preferably, the receiving support has a receiving surface for the culture plate of dark color, preferably black. This dark color of the support allows an accentuated contrast between the solid culture medium, which is generally transparent or translucent in the case of an agar, and said support, and prevents the formation of stray reflections on the screen.

Preferably, the display screen is a screen of bright color, preferably white. Again, this choice makes it possible to improve the quality of the two-dimensional images obtained on said screen.

Preferably, the photography means comprise a camera, said camera being preferably equipped with a band-pass filter centered on the wavelength of the incident light flux.

Preferably, the photography means are positioned facing the screen.

Again preferably, the photography means are positioned at a lower level than the means of projecting a light flux, preferably beneath said projection means. This results in a reduced footprint of the device.

In particular, preferably the means of projecting a light flux, the screen, and the photography means are mounted on a support frame and disposed above the support.

Preferably, the means of projecting a light flux are designed to produce a collimated light, preferably monochromatic.

Preferably, the means of projecting a light flux are means of laser type preferably comprising two laser diodes mounted in parallel. In particular, the means of projecting a light flux comprise at least one illumination element. This illumination element can be of laser type.

Preferably, the screen is retractable. This retraction, which can occur by winding of the screen or up and down displacement of the screen, makes it possible to further free up access to the support.

Preferably, the device comprises a communication and command module designed to control the photographing means and the photograph processing means as a function of instructions received from an inoculator control unit and to receive the results of the photograph processing means and transmit said results to said control unit. The presence of this module allows an automated operation, in connection with an inoculation facility.

The invention further relates to an automated facility for inoculation of a solid culture medium present in a layer on a culture plate, said facility comprising an inoculator of said culture plate, an inoculator control unit, and a device for detecting inoculation, characterized in that the control unit comprises instructions for inoculation, for photographing and for photograph processing and it is designed to control the inoculator as a function of these instructions, and in that the device for detecting inoculation comprises a communication and control module designed to communicate with the control unit and control the photographing means and the photograph processing means as a function of the instructions for photographing and photograph processing received from said control unit and to receive the results of the means of photograph processing and to transmit these results to said control unit.

Preferably, the instructions for inoculation, for photographing and for photograph processing are present in the form of sets of instructions with at least one of the sets corresponding to the following steps:
A) Verify the presence of the culture plate on the support;
B) Execute a step of inoculation with the aid of an inoculator;
C) Take a photograph with the aid of the photographing means;
D) Analyze the photograph with the aid of the photograph processing means.

The invention will be better understood upon perusal of the following description of sample embodiments, making reference to the enclosed drawings, in which:

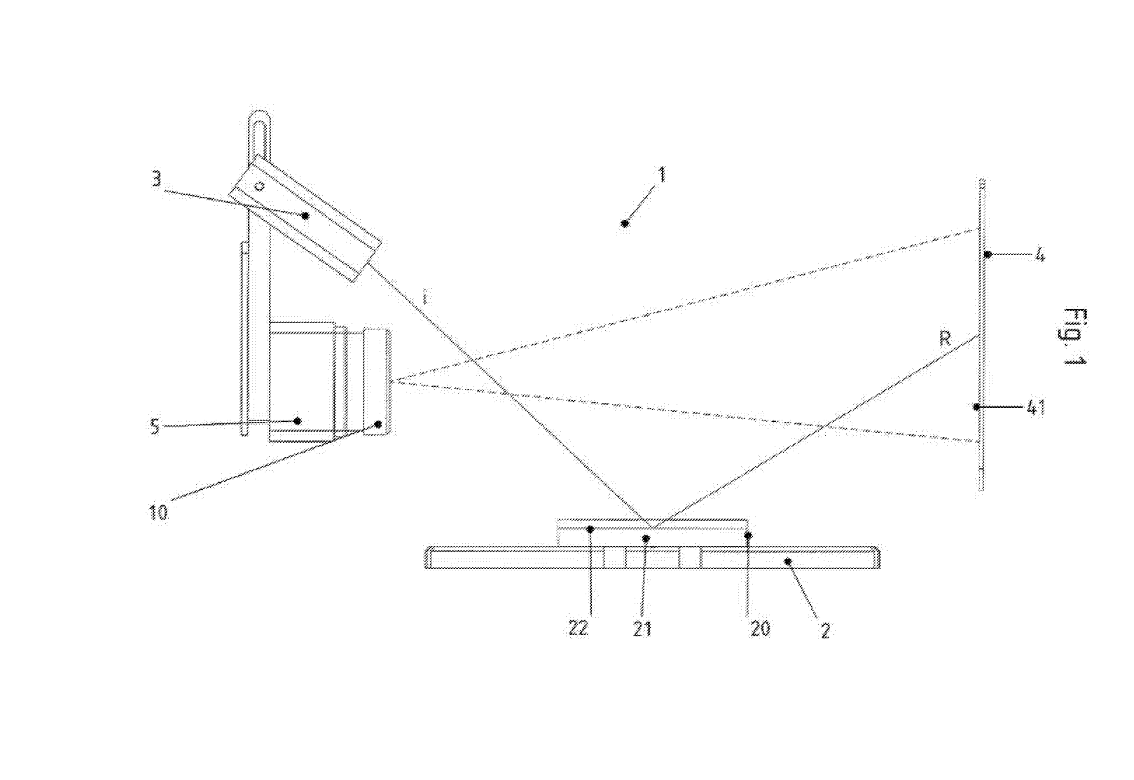

FIG. 1 represents a schematic diagram of the device for detecting inoculation according to the invention;

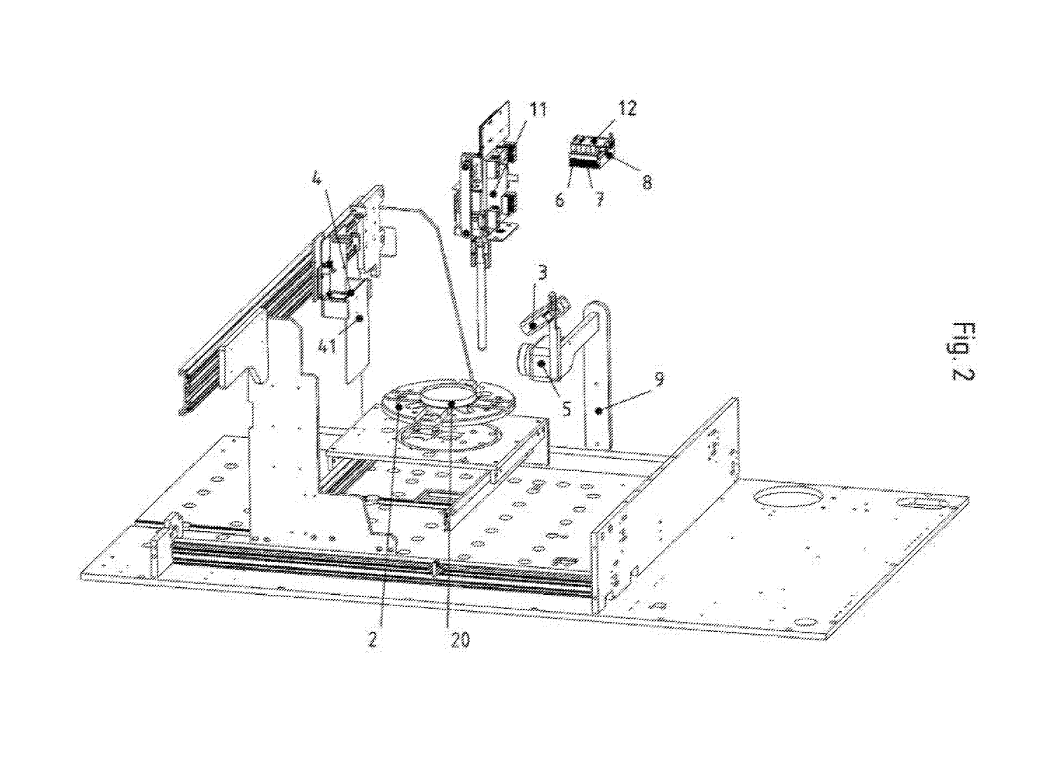

FIG. 2 represents a partial perspective view of a facility for inoculation equipped with a device for detecting inoculation according to the invention;

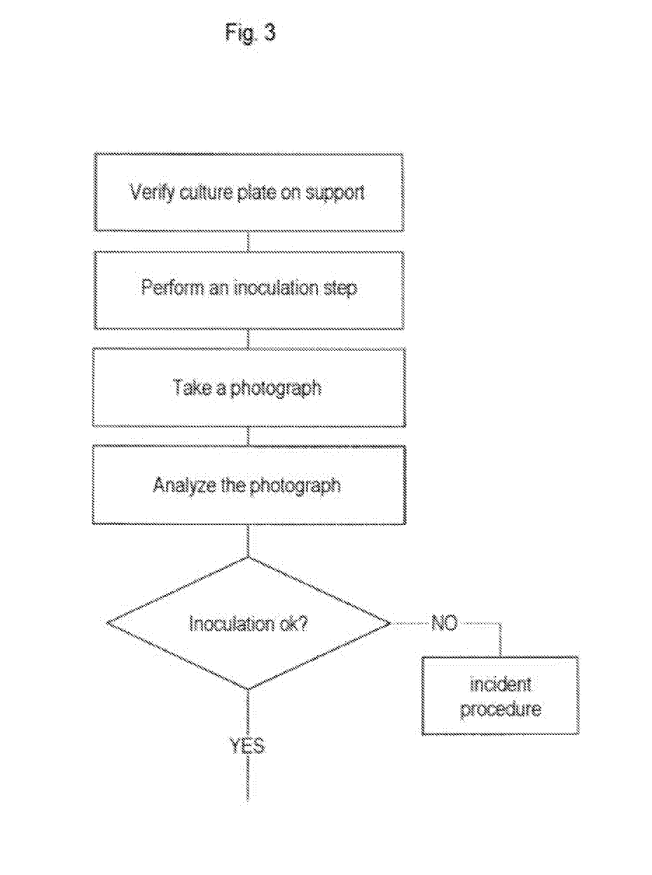

FIG. 3 represents schematically a portion of the steps of an inoculation program.

As mentioned above, the device 1 for detecting inoculation according to the invention is more particularly designed for the detecting of the inoculation with a given biological sample of a solid culture medium 21 present in a layer on a culture plate 20. The sample being analyzed may come from the food, the pharmaceutical, the cosmetic, or some other industry. This so-called biological sample is liable to contain living micro-organisms, whose presence and/or number are supposed to be analyzed, for example.

In the example shown, the culture plate 20 is formed by the body of a Petri dish, which is the most often encountered instance, even though other types of culture plates, generally having the shape of a dish with a rim, can be used.

In the example represented, the solid culture medium 21 is an agar medium formed of an agar poured into said Petri dish. The advantage of the agar medium is that once it solidifies it forms a layer of culture medium which is generally transparent or translucent, whose surface destined to form the inoculation surface 22 of the medium is smooth, this surface 22 extending substantially parallel to the midplane of the plate formed here by the bottom of the body of the dish.

The device 1 further comprises a support 2 to receive the culture plate 20 being inoculated. This support 2 is generally present in the form of a tray which may be outfitted with means of holding the culture plate on said support in a position in which the inoculation surface 22 of the culture medium of the plate extends substantially parallel to the midplane of said tray.

Generally, this support 2 is, as in the example represented, a rotary support revolving on itself, especially to facilitate the method of dial type isolation as mentioned above.

The receiving surface of the culture plate 20 of the support, formed in the case of a support of tray type by the top surface of the tray on which the culture plate 20 is intended to be placed, is of dark color. In the example represented, this receiving surface is black, to accentuate the contrast and facilitate the reflection of light.

In the example represented, the support 2 is integrated in an automated inoculation facility. This automated facility comprises driving means for the displacement of the culture plate 20 being inoculated for the positioning of the culture plate 20 on said support and means of lifting the culture plate 20 from said support. These means are not shown. The support 2 is furthermore represented here in the form of a support able to move up and down and in translation.

The inoculation facility thus comprises, in order to carry out the inoculation step, a support for the culture plate 20, this support being in common with the support of the culture plate of the device for detecting inoculation.

In the example represented, the inoculation facility also comprises an inoculator 11 of the culture plate 20 and a control unit 12 of the inoculator 11. This control unit 12 comprises instructions for inoculation, for photographing, and for photograph processing and it is designed to control the inoculator 11 as a function of these instructions.

Said control unit is preferably a system of electronic and/or computerized type, comprising for example a microcontroller or a microprocessor associated with a memory. Thus, when it is specified that said control unit, or means of said control unit, are designed to perform a particular operation, this means that the system comprises computer instructions and the corresponding means of execution to carry out that operation.

The inoculator 11 may involve a large number of shapes, since there are various automated inoculation facilities, as illustrated for example by the documents WO 98/41 610, WO 92/11 538 or U.S. Pat. No. 3,850,754.

In the example represented, this inoculator 11 comprises a storage zone for inoculation tools, not shown, a gripping device such as a forceps for grasping and driving the displacement of at least one inoculation tool, the control unit 12 being designed in particular to move the means for grasping and driving the displacement of the inoculation tool, namely, the gripping device, on the surface of the solid culture medium along a predefined trajectory corresponding to the inoculation instructions.

The control unit may comprise a plurality of sequences of inoculation instructions, each sequence being a function of the desired type of inoculation.

The control unit is thus designed to control the inoculator as a function of the selected inoculation instructions.

In order to enable the detection of the inoculation phase or phases as described above, the device for detecting inoculation comprises means 3 comprising at least one illumination element 3 for projecting an incident light flux toward the support 2, whose beam of incident rays forms a nonzero angle with the normal to said support, and in particular to the plane for receiving the culture plate 20 on said support 2 and therefore a nonzero angle with the normal to said inoculation surface 22 of the culture medium 21 of the culture plate 20, to allow an oblique illumination of the inoculation surface 22 of said culture medium 21 of the culture plate 20, in the state of the culture plate 20 placed on said support 2.

In the example represented, this angle between the beam of incident rays and the inoculation surface is close to 45°. Since the inoculation surface of the culture medium is a substantially planar smooth surface, the reflection of that surface can be approximated to a specular reflection. The rays reflected by said inoculation surface thus make an angle with the normal to said surface having a numerical value similar to that of the angle formed by the beam of incident rays with said normal.

In the example represented, the means for projecting a light flux comprise an illumination element of laser type, comprising two laser diodes mounted in parallel, in the manner of the optics of a pair of binoculars. These means of projecting a light flux are designed to produce a collimated, monochromatic light.

The detection device also comprises a display screen 4 having a display area 41 able to display a two-dimensional image of the incident light flux reflected by the inoculation surface 22 of the culture plate 20 in the state illuminated by the illumination element 3 projecting a light flux.

This display screen 4 is represented here in the form of a vertical screen which is retractable by movement up and down. This display screen could in similar fashion have been realized in the form of a windable screen. This display screen is a screen of light color, represented here in the form of a white screen. This screen is a nonreflecting screen, in order to distinguish it from a mirror. In the present case, the screen can be formed by a single sheet of paper. This screen is positioned at a higher level than the support 2 so as not to impede the movement of that support 2.

The device 1 for detecting inoculation also comprises means 5 of photographing the display area 41 of the screen. These photographing means 5 comprise a camera. This camera is equipped with a band-pass filter 10 whose chosen band frequency only allows the passage of the frequency band corresponding to that of the incident light flux produced by the means of projecting light.

Generally, the wavelength range used is around 650 nm, which is the wavelength of the incident light.

In the example represented, the photographing means are positioned facing the screen, at a lower level than the illumination element 3, in particular beneath the illumination element 3.

The illumination element 3, the screen 4 and the photographing means 5 are mounted on a support frame 9 and disposed above said support 2. At least the photographing means 5 are mounted such that their position on the support frame 9 can be adjusted in order to optimize the photographing.

The detection device 1 also comprises a memory 7 for saving said photographs of the display area 41 of the screen 4 and means 8 for processing said photographs as a function of reference data in order to determine the state of inoculation. These processing means 8 comprise a processor, in the embodiments described below.

Finally, the detection device 1 comprises a communication and control module 6 designed to communicate with the control unit 12 and control the photographing means 5 and the photograph processing means 8 as a function of instructions for the photographing and the photograph processing received from said control unit 12, and to receive the results of the photograph processing means 8 and transmit said results to said control unit 12.

Once again, the communication and control module 6 is a system of electronic and/or computerized type, comprising for example a microcontroller or a microprocessor associated with a memory. Thus, when it is specified that the module, or means of said module, are designed to perform a particular operation, this means that the system comprises computer instructions and the corresponding means of execution to carry out that operation.

Generally speaking, the instructions for inoculation, for photographing and for photograph processing are present in the form of sets of instructions with at least one of the sets corresponding to the following steps:
A) Verify the presence of the culture plate 20 on the support 2;
B) Execute a step of inoculation with the aid of the inoculator 11;
C) Take a photograph with the aid of the photographing means 5;
D) Analyze the photograph with the aid of the photograph processing means 8.

The inoculation step may involve a step of placement with the aid of the inoculation tool of a drop of biological sample being inoculated on the inoculation surface.

The inoculation step may involve a step of sweeping the inoculation surface with the aid of the inoculation tool, starting from the surface of the zone of placement of the drop.

As a variant, the inoculation step may involve a simultaneous step of placement of the sample and sweeping the inoculation surface with the aid of the inoculation tool, loaded with sample.

As a function of the instructions for photographing and processing received by the means of photographing and photograph processing, these latter can be controlled to take a photograph prior to the deposition. In this case, the photograph taken corresponds to the inoculation surface prior to any inoculation operation.

The means of photographing and photograph processing can likewise be controlled based on instructions for photographing and photograph processing received in order to take a photograph after the depositing of the drop. In this case, the processor for the photograph processing which is designed to process that photograph in order to determine whether the state of the inoculation is OK or not OK may be designed in variable manner according to whether the zone of depositing of the sample, known as a drop, is on the periphery or in the interior of the image formed by the reflection of the agar. In all cases, this photograph processing performed by the processor is generally based on an analysis of gray levels of at least a portion of the photograph. Thus, this processor for the photograph processing comprises analysis means, also known as a gray level analyzer, for at least one portion of a photograph, and generally means for defining a region of interest forming the portion of the photograph being analyzed in gray levels. The definition of the region of interest is variable depending on the type of inoculation and the type of gray level analysis. In the case when the image processing is meant to validate the depositing of the sample drop, this processor for the image processing comprises means of defining a region of interest forming that portion of the photograph to be analyzed. This region of interest generally corresponds to a zone of the photograph with a surface higher than the surface presumed to be covered by the drop of sample deposited and having covered that surface at least partly. In the first case described below, this region of interest has an area at least equal to three times the presumed area of the surface occupied by the drop of sample deposited. In the second case described below, the region of interest may have an area substantially equal to the presumed area of the surface occupied by the drop.

Thus, if the drop is at the periphery of the image formed by the reflection of the agar, one can arrange to detect the transitions of gray levels in the region of interest corresponding to the border of the drop in the photograph and then do a linear and then a circular interpolation of points detected in order to identify the absence or the presence of the drop.

If the drop is on the inside of the image formed by the reflection of the agar, one can arrange to:
measure the gray intensities of each pixel of the photograph in the region of interest (that is, the region of the photograph assumed to contain the drop), the location of this zone of interest forming a reference data point;
take the average of these gray intensities in said region of interest;
compare the average of said gray intensities to a threshold value forming a reference data point;
transmit the result of the comparison via the communication module to the control unit.

In other words, in this second case, since the reference data points comprise location data corresponding to the assumed location of the drop in the photograph known as the region of interest and a gray level threshold value, the photograph processing means may comprise means of measuring the gray level of the zone of the region of interest, means of comparison of the measured value against the reference threshold value, and means of determining whether the state of the inoculation is OK or not OK, depending on the result of the comparison. The OK result corresponds to the presence of a dark spot formed by the drop in said region of interest.

If the result of the comparison is such that the measured value of the gray averages is greater than the reference threshold value, the control unit is designed to continue executing the inoculation program as initially scheduled.

If the result of the comparison is such that the measured value of the gray averages is less than the reference threshold value, the control unit is designed to launch an incident procedure which may contain a large number of forms of execution. Thus, this incident procedure may involve a stoppage of the facility or a repeating of the inoculation step not properly performed. Thus, this incident procedure will not be described in detail.

In the event that the inoculation program is continued, the means of photographing and photograph processing can be commanded by instructions for photographing and photograph processing to take a photograph after sweeping the inoculation surface with the aid of the inoculation tool and formation of streaks on said surface. To facilitate the image processing, it is preferable for the streaks to extend vertically in the photograph, that is, in the image captured by the photographing means. In order to obtain such a result, it is possible to orient the support 2 appropriately with respect to the photographing means 5, which is easy to do when the support is rotational.

The further processing of the photograph to detect the streaks may involve, for example:

the definition of a region of interest where the streaks are assumed to be present.

the vertical projection of the values of gray levels onto a horizontal axis.

the obtaining of a profile whose values correspond to the sum of the gray level values along the vertical axis of the image.

The profile obtained is a representation in one dimension of the region of interest of the photograph, which itself is in two dimensions. On this profile, the bright portions are the peaks, the dark portions are the valleys. By analyzing the derivative of the profile, it is possible to detect the transitions peak to valley (bright to dark), or valley to peak (dark to bright) and thus detect the presence or absence of streaks. A minimum number of streaks is adjusted in order to eliminate artifacts in the photograph.

Of course, once again, other embodiments of the gray level analysis of the region of interest can be contemplated without leaving the scope of the invention.

The procedures described above apply when the inoculation is done in two steps, in the first step with a depositing of a drop of sample using the inoculation tool prior to the step of spreading of the drop over the inoculation surface using the inoculation tool. Thus, the photograph prior to inoculation makes it possible to visualize the state of the agar surface prior to inoculation. The photograph during the inoculation makes it possible to be assured of the depositing of the sample by the appearance of a dark spot on the photograph, in a location corresponding to the zone of depositing of the drop. The photograph after inoculation makes it possible to visualize brighter features corresponding to the streaks formed by the sweeping of the inoculation surface with the aid of the inoculation tool.

When the inoculation tool is a swab, the photograph during the inoculation is omitted, since there is no step of depositing a drop that is distinct from the sweeping step.

In certain cases, the step of photographing prior to inoculation is likewise omitted.

The skilled person will easily understand that the different steps, notably the steps performed by the communication module 6, the processing means 8 and the control unit 12, depending on the embodiments presented above, can be performed in the form of sets of computer instructions implemented in a processor or they can be realized by dedicated electronic components or components of FPGA or ASIC type. It is also possible to combine computer parts and electronic parts. These computer programs or computer instructions may be contained in program storage devices, such as digital data storage media which can be read by computer or executable programs. The programs or instructions can likewise be executed from program storage peripherals.

At the end of the inoculation, the memorized photograph(s) is (are) memorized in correlation with the data pertaining to the sample or to the inoculated culture plate.

For this purpose, the facility may include, in familiar fashion, means of identification of a marking of the sample and/or the culture plate, these identification means being connected to the control unit to provide that unit with information concerning said marking.

1. A method for detecting inoculation in order to detect inoculation with a sample of a solid culture medium present in a layer on a culture plate, said culture plate being positioned on a support,
wherein said method includes a step of projecting an incident light flux towards said support in order to allow oblique illumination of the surface referred to as the inoculation surface of said culture medium of the culture plate when the culture plate is provided in the disposed state on said support, a step of displaying on a display area of a display screen of a two-dimensional image of the incident light flux reflected by the inoculation surface of the culture plate in the illuminated state illuminated by the means for projecting a light flux comprising at least one illumination element and a step of photographing the display area of the screen, each photograph being able to be processed to determine the inoculation state of said culture medium.

2. A device for detecting inoculation in order to detect inoculation with a sample of a solid culture medium present in a layer on a culture plate, wherein the device comprises a support to receive the culture plate being inoculated, means of projecting an incident light flux towards said support in order to allow oblique illumination of the surface referred to as the inoculation surface of said culture medium of the culture plate when the culture plate is in the disposed state on said support, a display screen comprising a display area able to display a two-dimensional image of the incident light flux reflected by the inoculation surface of the culture plate in the state illuminated by the means of projecting a light flux, and means of photographing the display area of the screen.

3. The device as claimed in claim 2, wherein said device comprises a memory for storing said photographs of the display area of the screen and means of processing said photographs in order to determine the state of inoculation.

4. The device as claimed in claim 2, wherein the receiving support has a receiving surface for the culture plate of dark color, preferably black.

5. The device as claimed in claim 2, the display screen is a screen of bright color, preferably white.

6. The device as claimed in claim 2, wherein the photography means comprise a camera, said camera being preferably equipped with a band-pass filter centered on the wavelength of the incident light flux.

7. The device as claimed in claim 2, wherein the photography means are positioned facing the screen.

8. The device as claimed in claim 2, wherein the photography means are positioned at a lower level than the means of projecting a light flux, preferably beneath said projection means.

9. The device as claimed in claim 2, wherein the means of projecting a light flux, the screen and the photography means are mounted on a support frame and disposed above the support.

10. The device as claimed in claim 2, wherein the means of projecting a light flux are designed to produce a collimated light, preferably monochromatic.

11. The device as claimed in claim 2, wherein the means of projecting a light flux are means of laser type preferably comprising two laser diodes mounted in parallel.

12. The device as claimed in claim 2, wherein the screen is retractable.

13. The device as claimed in claim 3, wherein comprises a communication and command module designed to control the photographing means and the photograph processing means as a function of instructions received from a control unit of inoculation means and to receive the results of the photograph processing means and transmit said results to said control unit.

14. An automated facility for inoculation of a solid culture medium present in a layer on a culture plate, said facility comprising an inoculator of said culture plate, an inoculators control unit, and a device for detecting inoculation, wherein the control unit comprises instructions for inoculation, for photographing and for photograph processing and it is designed to control the inoculator as a function of these instructions, and in that the device for detecting inoculation is in accordance with claim 3, and comprises a communication and control module designed to communicate with the control unit and control the photographing means and the photograph processing means as a function of the instructions for photographing and photograph processing received from said control unit and to receive the results of the means of photograph processing and to transmit these results to said control unit.

15. The facility as claimed in claim 14, wherein the instructions for inoculation, for photographing and for photograph processing are present in the form of sets of instructions with at least one of the sets corresponding to the following steps:
A) Verify the presence of the culture plate on the support;
B) Execute a step of inoculation with the aid of the inoculator;
C) Take a photograph with the aid of the photographing means;
D) Analyze the photograph with the aid of the photograph processing means.
